(19) United States
(12) Patent Application Publication
(10) Pub. No.: US 2012/0302604 A1
(43) Pub. Date: Nov. 29, 2012

(54) Modulation of Phospholipase D for the Treatment of the Acute and Chronic Effects of Ethanol

(76) Inventors: Gilbert DiPaolo, Northford, CT (US); Tiago Gil Oliveira, Guimaraes (PT); Samuel G. Frere, Pabu (FR); Markus Wenk, Singapore (SG); Robin Chan, Singapore (SG)

(21) Appl. No.: 13/359,350

(22) Filed: Jan. 26, 2012

Related U.S. Application Data
(63) Continuation of application No. PCT/US2010/043659, filed on Jul. 29, 2010.
(60) Provisional application No. 61/230,502, filed on Jul. 31, 2009.

(51) Int. Cl. A61K 31/454; A61P 1/08; G01N 27/62; A61P 9/00; A61P 9/10; A61P 1/16; A61P 25/28; A61P 9/12

(52) U.S. Cl. 514/322; 435/21

(57) ABSTRACT

The present invention relates to methods of decreasing the negative effects of alcohol on behavior as well as inhibiting the toxic effects of alcohol, comprising administering, to a subject, an effective amount of an inhibitor of phospholipase D.

### PRIORITY CLAIM

This application is a continuation of International Patent Application No. PCT/US2010/043659, filed Jul. 29, 2010, which claims priority to U.S. Provisional Application Ser. No. 61/230,502, filed Jul. 31, 2009, to both of which priority is claimed and the contents of both of which are incorporated herein in their entireties.

### GRANT INFORMATION

This invention was made with government support under R01 NS056049 and R21 AA015525 awarded by the National Institutes of Health. The government has certain rights in the invention.

### 1. INTRODUCTION

The present invention relates to methods of decreasing the negative effects of ethanol on behavior as well as inhibiting the toxic effects of ethanol, comprising administering, to a subject, an effective amount of an inhibitor of phospholipase D (“PLD”).

### 2. BACKGROUND OF THE INVENTION

Alcoholism is a complex and common disorder characterized by excessive consumption of ethanol, the development of tolerance and dependence, and the impairment of social and occupational functioning (1). Despite the burden of alcoholism for our society, available treatments are limited and many patients relapse, demonstrating the necessity for the development of new therapeutics.

While cellular membranes are known to be primary targets of ethanol, the neurobehaviorally-relevant lipid changes induced by this drug have remained elusive. The proposal that PLD may mediate some of the effects of alcohol arose following the identification of phosphatidylethanol (PEtOH) in organs of ethanol-treated rats, and the recognition that PLD activity is responsible for PEtOH synthesis (2). Indeed, while PLD enzymes hydrolyze phosphatidylcholine (PC) to produce choline and the bioactive lipid phosphatidic acid (PA) under normal conditions, they prefer ethanol over water by a multiple of one thousand, leading to the preferential synthesis of PEtOH at the expense of PA in the presence of ethanol. This phenomenon has been broadly utilized to measure PLD activity in cells (2, 3). The unique properties of PEtOH, including its high resistance to lipases, cause this lipid to rapidly accumulate in ethanol-exposed tissues, particularly the brain, where it may alter the physicochemical and signaling properties of neuronal membranes (1, 2). Despite the fact that PLD enzymes are primary targets of ethanol, there is no experimental evidence supporting a role for this pathway in mediating the effects of this drug.

### 3. SUMMARY OF THE INVENTION

The present invention relates to methods of decreasing the negative effects of ethanol on behavior as well as inhibiting the toxic effects of ethanol, comprising administering, to a subject, an effective amount of an inhibitor of phospholipase D (“PLD”), which may be an inhibitor of PLD1 and/or PLD2. It is based, at least in part, on the discovery that mice genetically engineered to lack PLD2 (PLD2 “knockout” or “PLD2KO” mice) as well as “double knockout” mice engineered to lack both PLD1 and PLD2 were more resistant to the negative effects of ethanol compared to their wild type counterparts.

Accordingly, in particular non-limiting embodiments, the present invention provides for a method of decreasing a negative effect of ethanol intake on behavior comprising administering, to a subject in need of such treatment, an effective amount of a PLD inhibitor. Such methods may be useful in the management of acute intoxication as well as in rehabilitation from alcohol addiction. Unless specified otherwise, the term “alcohol” as used herein refers to ethanol.

In further non-limiting embodiments, the present invention provides for a method of inhibiting the toxic effects of ethanol on a cell or tissue, for example, but not limited to, the brain, liver, and/or pancreas and/or a brain cell, a liver cell, and/or a pancreatic cell. Such methods may be useful in the treatment of acute toxic effects on these tissues and/or the chronic effects of alcohol abuse.

In still further non-limiting embodiments, the present invention provides for a method of detecting ethanol exposure of a human subject comprising testing a sample from the subject for the presence of phosphatidylethanol (“PEtOH”), where the presence of PetOH in the sample indicates that the subject was exposed to ethanol Further, by measuring the amount of PEtOH, the extent of ethanol exposure may be evaluated by comparison with control values.

In related non-limiting embodiments, the present invention provides for a method of detecting and/or monitoring PLD activity in a cell, a tissue, and/or a subject comprising (i) administering ethanol to the cell, tissue or subject and (ii) detecting and/or measuring PEtOH in a sample from the cell, tissue or subject, comprising subjecting the sample to mass spectrometry or liquid chromatography-mass spectrometry (“LC-MS”), wherein the presence and optionally the quantity of PEtOH reflects the PLD activity present in the cell, tissue, or subject. Species of PEtOH detectable by LC-MS vary according to the fatty acyl composition. LC-MS may be used, for example, to evaluate the effects of a PLD inhibitor on PEtOH production, to identify a compound having PLD inhibitory activity, and/or to screen tissues (e.g. in subjects treated with/self-administering ethanol) for sites of modulated PLD activity.

### 4. BRIEF DESCRIPTION OF THE FIGURES

FIG. 1. The transphosphatidylation reaction mediated by PLD. In the presence of water, PLD generates phosphatidic acid (PA) by hydrolyzing phosphatidylcholine (PC). However, in the presence of ethanol, they generate phosphatidylethanol (PEtOH) which may have toxic effects when it accumulates in tissues, such as brain and liver.

FIG. 2A-C. PLD2KO mice recovered faster from ethanol injections in the rotarod test. Mice were placed on the rotarod cylinder, the speed of which was gradually accelerated from 3 to 35 revolutions per minute over a 6 minute period. Latentcy to fall off the cylinder was recorded. On day 1, ethanol naive mice (WT, n=13; PLD2KO, n=12) were trained to remain on the accelerating rotarod (4 trials of 6 minutes). (A.) On day 2, the fall latency was measured for the trained mice (1 trial of 6 minutes). No difference was found between WT and PLD2KO mice. (B.) Mice were then injected with ethanol (3 g/kg i.p.), and tested for recovery of balance on the accelerating rotarod at 3, 30, 60 and 120 minutes after injection. The time course graph represents the latency to fall at the different times after injection normalized to the latency time obtained before injection. Statistical analysis was done using the two-way ANOVA test.(C) Results of rotarod test in double knockout mice lacking PLD1 and PLD2, as compared to wild-type. WT males (n=11) and females (n=12), Pld1−//Pld2−/− males (n=12) and females (n=12). Pld1−//Pld2−/− mice are referred to as double knockout or DKO. Values denote means±S.E.M.

FIG. 3. PLD2KO mice are less sensitive to ethanol injections in the loss of righting reflex test.

FIG. 4A-B. Ethanol-injected PLD2KO mice have a reduced ability to synthesize phosphatidylethanol (PEtOH) in the brain (A) and in the liver (B). The white bars represent PEtOH levels measured in tissue from ethanol-injected wild type mice, while the black bars represent PEtOH levels obtained in tissue from ethanol-injected PLD2KO animals. Vehicle-injected animals showed background levels of PEtOH, as expected. The data show decreased PEtOH levels in PLD2 KO tissue relative to wild type tissue upon exposure ton ethanol.

FIG. 5A-C. (A) Detection and quantification of brain lipid substrates and products of PLD enzymatic pathway. Brain lipids were extracted from Pld2+/+ and Pld2−/− mice, with and without ethanol injection and subjected to LC/MS analysis. Four major PEtOH species were detected and presented together with their corresponding glycerophospholipids PC and PA and neutral lipid DAG species. All data are mean±s.d. The differences in PEtOH levels were analyzed using paired t-test and significance (p<0.01) is indicated with (**). Graph legend: white bars, ethanol-injected WT mice (n=7); black bars, ethanol-injected Pld2−/− mice (n=9); light gray bars, vehicle-injected WT mice (n=7); and light gray bars, vehicle-injected Pld2−/− mice (n=6). (B) Left panel, Pre-injection trial: trained WT mice (n=16) and Pld2−/− (n=15) littermate perform similarly in the accelerating rotarod (p>0.05). Right panel, Vehicle control: Pld2+/+ (n=8) and Pld2−/− (n=6) mice perform similarly on the rotarod 3 min after i.p. injection of vehicle (p>0.05) (C) Post-injection recovery: the latency to fall from the accelerating rotarod was measured at 3 and 60 min after peritoneal ethanol-injection of WT (n−16) and Pld2−/− (n=15) mice. The recovery from ataxia is significantly faster for the Pld2−/− mice relative to controls (repeated measure two-way ANOVA test, p<0.05 for the genotypes, F(1,29)=4.4). One hour after the injection, Pld2−/− mice spent more time on the rotarod than their WT littermates. Values denote means±S.E.M, after normalization to TO values (i.e., the animals' performance prior to the injection of ethanol, which is similar for both genotypes).

FIG. 6A-J. (A) Gene targeting strategy showing the Cre-mediated ablation of exon 13-15 from the Pld2 gene. See experimental procedures. (B) Western blot analysis showing the lack of PLD2 immunoreactivity in adult brain from Pld2−/− mice. The expression of a variety of control proteins is not affected by the ablation of PLD2. (J) Lipid profile of mice brain lipid extracts. Glycerophospholipids and sphingolipids were analyzed by LC/MRM using gradient elution and signal levels were converted to relative abundance levels by normalization to spiked internal standards. Neutral lipids were analyzed by LC/Q3 using isocratic elution and converted to relative abundance levels by normalization to total signal. All data are mean±s.d. Graph legend: white bars, ethanol-injected WT mice (n=7); black bars, ethanol-injected Pld2−/− mice (n=9); light gray bars, vehicle-injected WT mice (n=7); and light gray bars, vehicle-injected Pld2−/− mice (n=6). Phosphatidylserine, PS; Phosphatidylinositol, PI; Phosphatidylethanolamine, PE (a-diacyl linkage; p-1′-alkenyl-2-acyl linkage); Phosphatidylcholine, PC; Phosphatidic Acid, PA; Sphingomyelin, SM; Ceramide, Cer; Glucosylceramide, GluCer; Ganglioside, GM3; Triacylglycerol, TAG; Diacylglycerol, DAG.

FIG. 7A-M. PLD inhibitors.

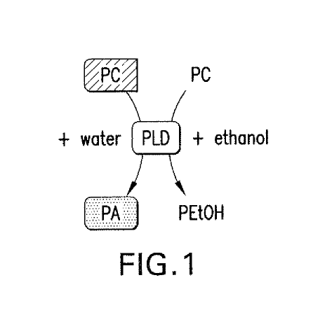

### 5. DETAILED DESCRIPTION OF THE INVENTION

For purposes of clarity, and not by way of limitation, the detailed description of the invention is divided into the following subsections:

(i) PLD inhibitors;

(ii) ethanol-related disorders; and

(iii) methods of treatment.

5.1 PLD Inhibitors

Inhibitors of PLD activity, including PLD1 and PLD2 inhibitors, may be used according to the invention. A PLD inhibitor decreases the amount of PLD activity present in the subject to which it is administered, and may do so by any mechanism, including direct inhibition of enzyme activity as well as reduction in the amount or availability of PLD.

In certain non-limiting embodiments, the invention provides for the use of an agent that inhibits the enzyme activity of PLD, which may be PLD1 and/or PLD2, although inhibition of PLD2 is preferred.

In one non-limiting embodiment, an agent that inhibits PLD including PLD2 is 5-Fluoro-2-indolyl des-chlorohalopemide (“FIPI”).

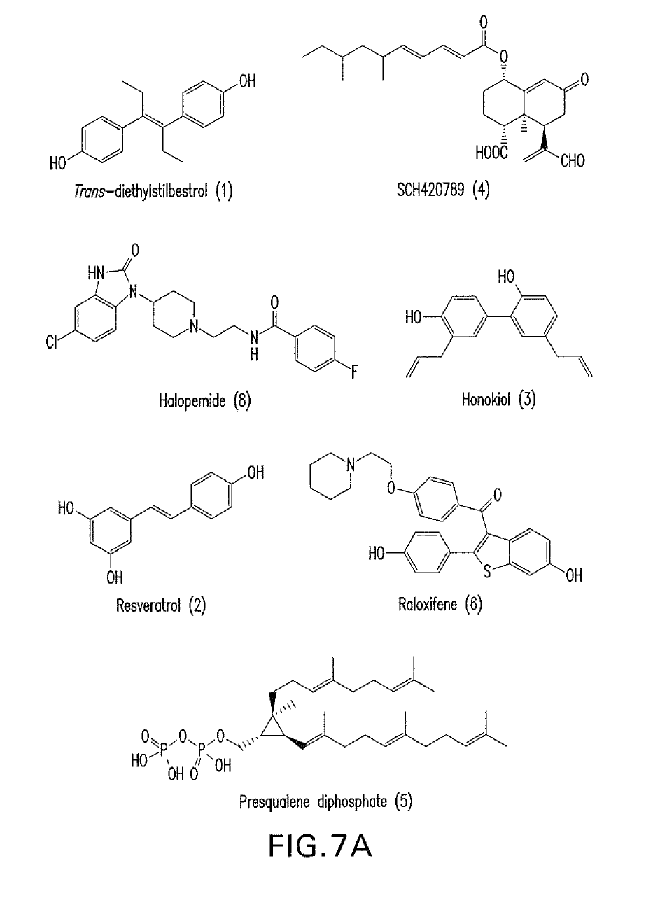

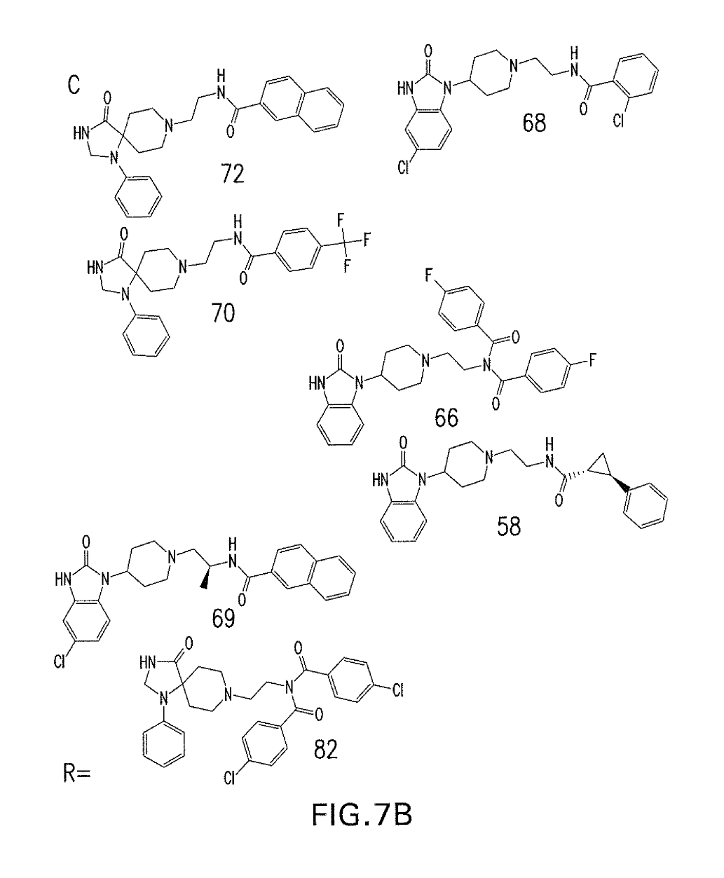

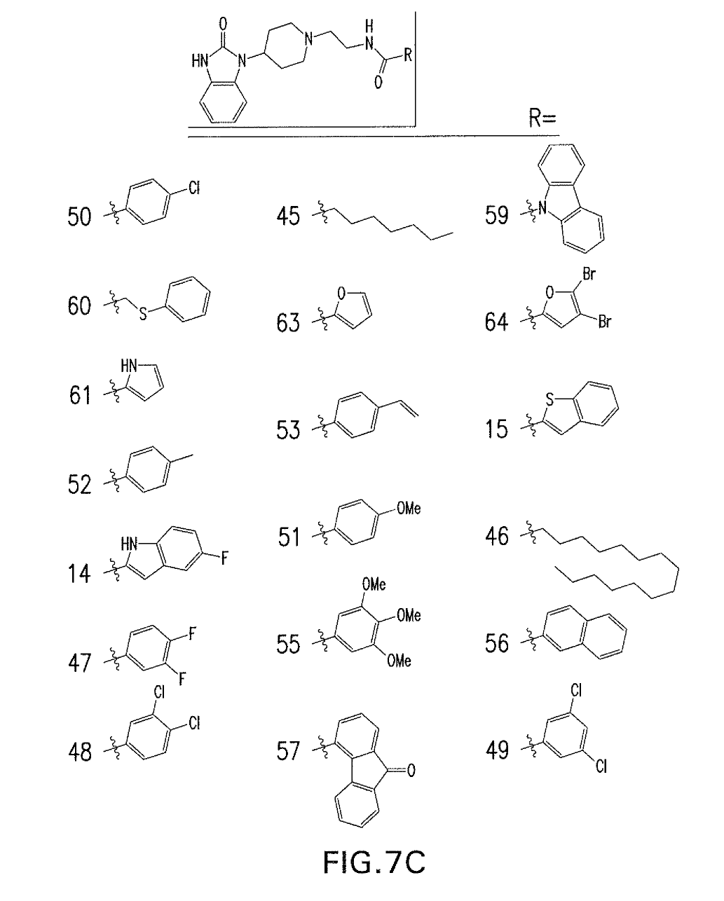

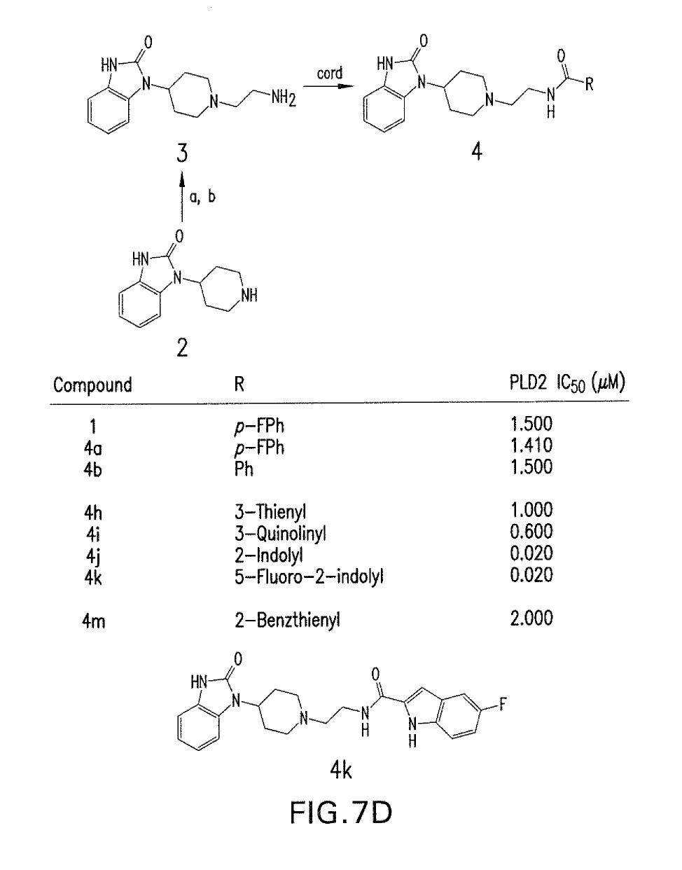

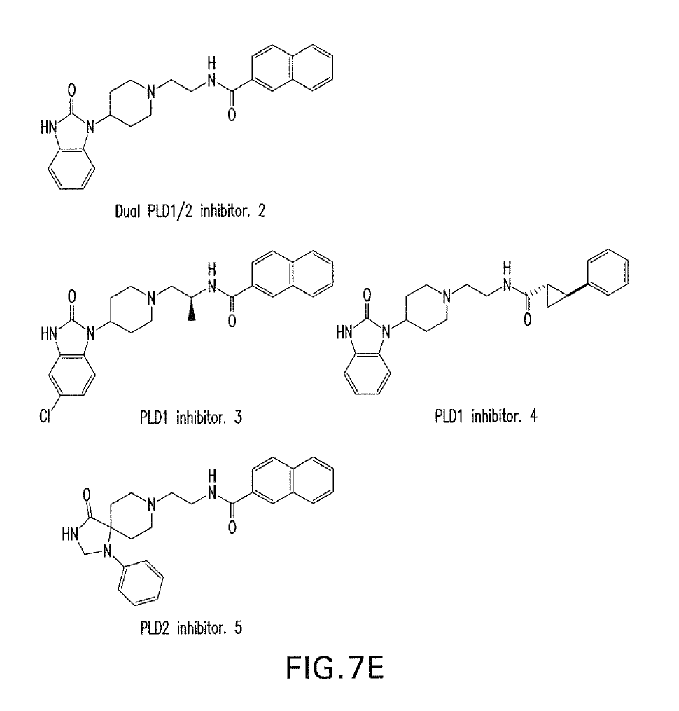

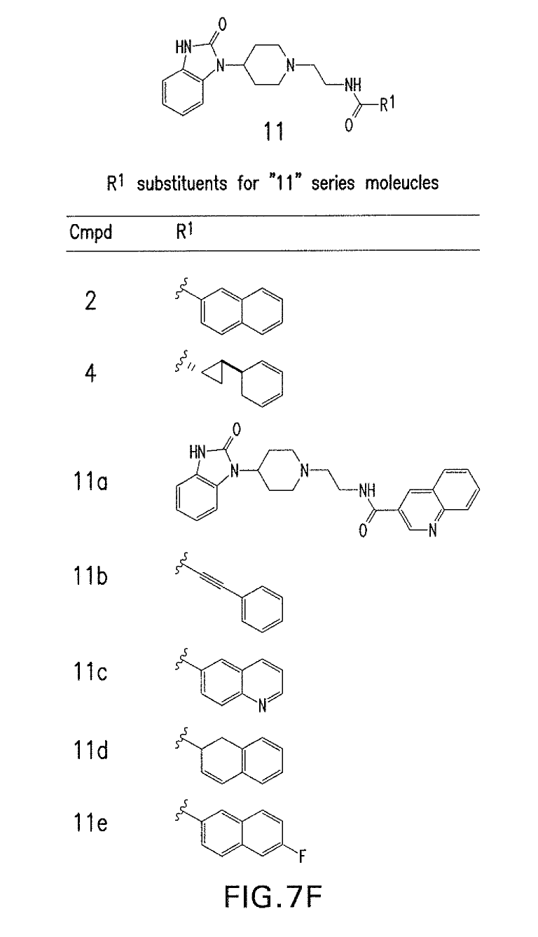

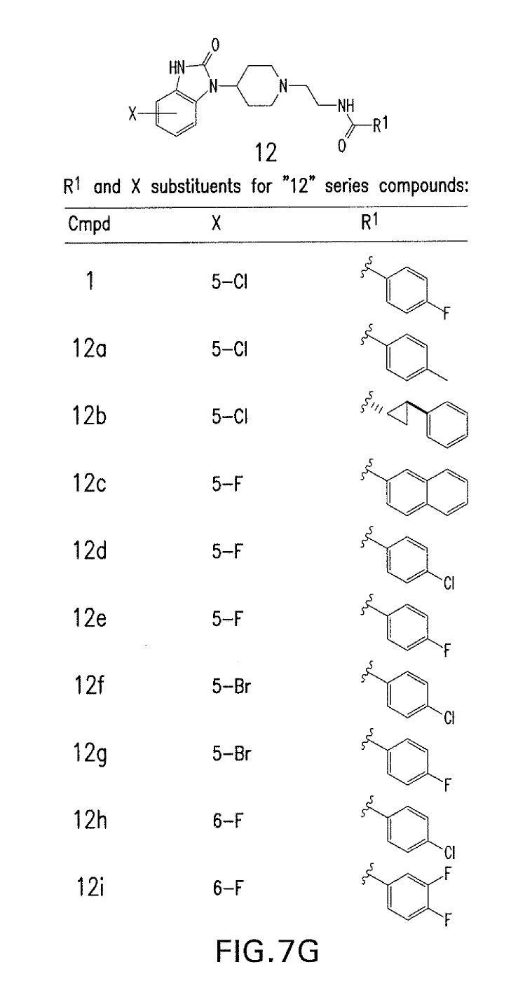

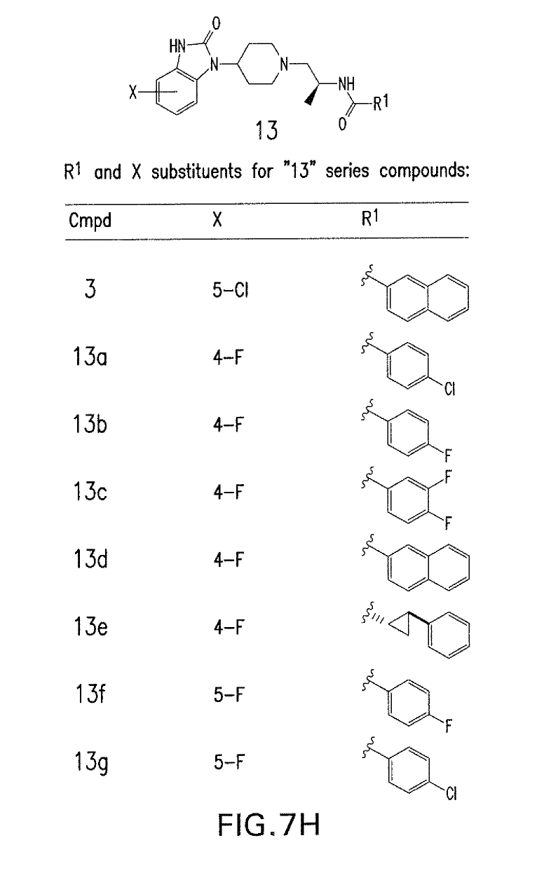

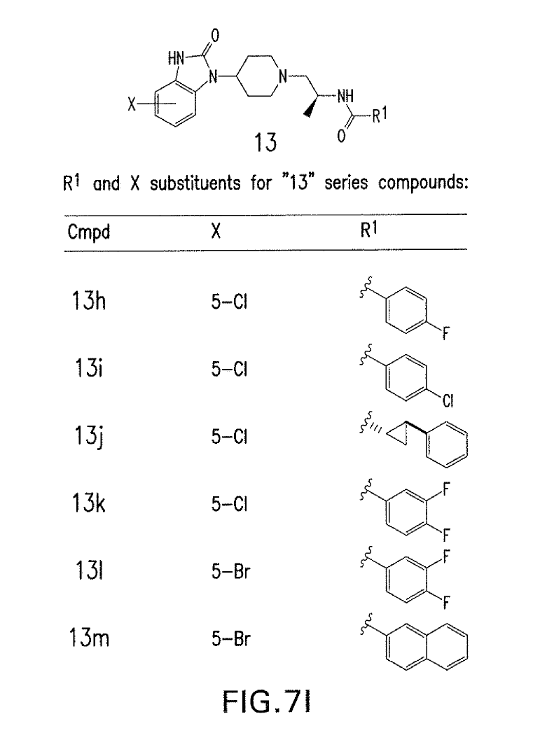

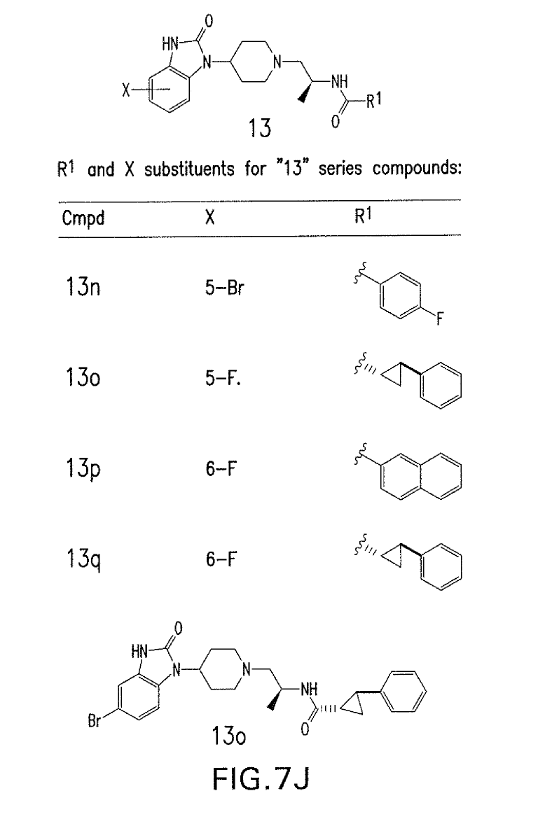

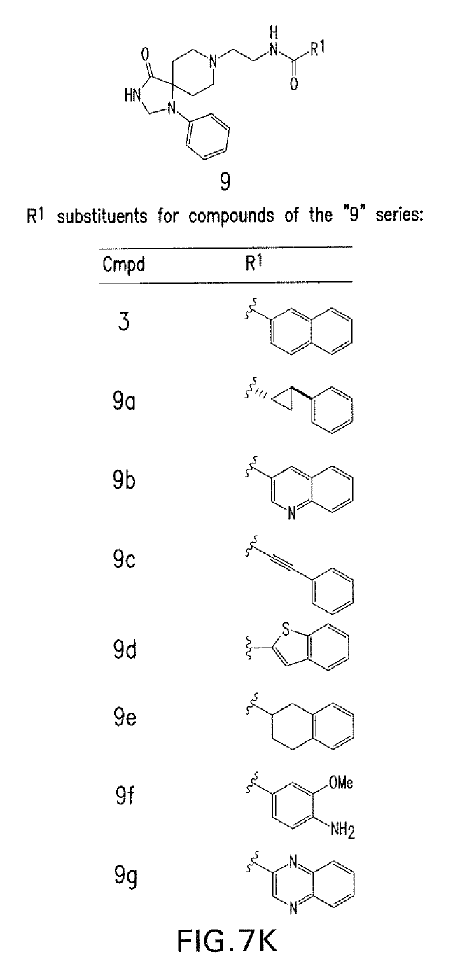

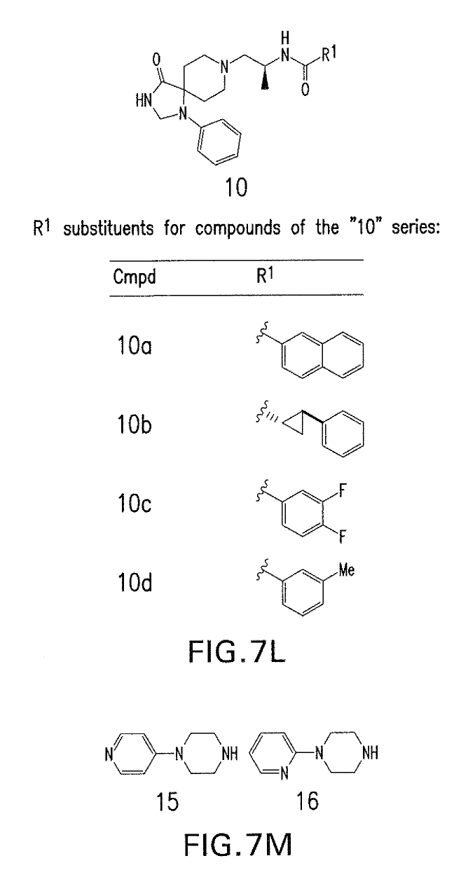

Additional PLD inhibitors, which may be used according to the invention include, but are not limited to: diethylstibestrol, resveratrol, honkiol, SCH420789, presqualene diphosphate, raloxifene, halopemide, 4-hydroxy tamoxifen, compounds depicted in FIGS. 7A-C (Scott et al., 2009, Nat Chem Bial 5(2):108-117 and its supplemental information online at Nature Chemical Biology 10.1038/nchembio.140); halopemide derivatives, especially halopemide derivatives comprising a 2-indolyl moiety, including compounds set forth in FIG. 7D (Monovich et al., 2007, Bioorg. Med. Chem. Lett. 17:2310-2311); compounds shown in FIGS. 7E-J including, but not limited to, halopemide derivatives comprising a halogenated piperidinyl benzimidazolone moiety and a S-methyl moiety (Lewis et al., 2009, Bioorg. Med. Chem. Letts. 19:1916-1920); derivatives of compound 5 of FIG. 7E, including compounds that comprise a 1,3,8-triazaspiro[4,5]decan-4-one structure, including compounds depicted in FIG. 7K-L (Lavieri et al., 2009, Bioorg. Med. Chem. Lett, 19:2240-2243); and compounds depicted in FIG. 7M (Lavieri et al., 2009, Bioorg. Med. Chem. Lett. 19:2240-2243).

In particular, preferred non-limiting embodiments, the PLD inhibitor is a PLD2 selective inhibitor such as, but not limited to, 4-OH tamoxifen; compounds 72 and 82 of FIG. 7B (Scott et al., 2009, Nat Chem Biol 5(2):108-117); compounds 4j and 4k of FIG. 7D (Monovich et al., 2007, Bioorg. Med. Chem. Lett. 17:2310-2311); compound 5 of FIG. 7E (Lewis et al., 2009, Bioorg. Med. Chem. Letts. 19:1916-1920); and derivatives of compound 5 of FIG. 7E, including compounds that comprise a 1,3,8-triazaspiro[4,5]decan-4-one structure, including compounds depicted in FIG. 7K-L (Lavieri et al., 2009, Bioorg. Med. Chem. Lett. 19:2240-2243). Each of the foregoing references and any publicly supplied supplemental information linked thereto, and any compounds and/or synthetic schemes set forth therein, are incorporated by reference in their entireties herein.

Additional PLD inhibitors may be identified by methods known in the art, including, but not limited to, the assays set forth in Scott et al., 2009, Nat Chem Biol. February;5(2):108-17; Monovich et al., 2007, Bioorg. Med. Chem. Lett. 17:2310-2311; Lewis et al., 2009, Bioorg. Med. Chem. Letts. 19:1916-1920; or Lavieri et al., 2009, Bioorg. Med. Chem. Lett. 19:2240-2243.

Alternatively, a PLD inhibitor may be a molecule which decreases expression of PLD, and especially PLD2, for example a small interfering RNA or an antisense RNA comprising a portion complementary to the PLD1 and/or PLD2 gene.

For example, and not by way of limitation, the present invention provides for a method of determining that a test compound is a PLD inhibitor, comprising administering the test compound, to a tissue or subject that manifests PLD activity (which may be PLD1 and/or PLD2 activity), in the presence of ethanol (which may be present prior to administration of the test compound, after administration of the test compound, or may be co-administered with the test compound) and then determining whether the test compound inhibits production of PEtOH (for example, by comparison with a pre-established production level or with a suitable control). Production of PEtOH may be evaluated using any method known in the art including, but not limited to, LC-MS.

5.2 Ethanol-Related Disorders Ethanol-related disorders, the manifestations of the negative and toxic effects of ethanol intake, which may be treated according to the invention include, but are not limited to, the behavioral effects of intoxication (i.e., negative effects on behavior) including, but not limited to, impaired motor ability, including impaired coordination (including ataxia), impaired cognition, vertigo, nausea, and anterograde amnesia; and other negative effects on behavior including but not limited to substance abuse, addiction, and symptoms and signs of detoxification; conditions associated with toxic effects of alcohol, including but not limited to neurodegeneration (e.g. cerebellar degeneration); seizure; neuropathy; hypertension; cardiovascular disease; cardiomyopathy; cardiac angina; myocardial infarction; vascular disease; cerebral ischemia; cerebral infarction; liver disease including but not limited to acute and chronic hepatitis, fatty liver, hepatic fibrosis, cirrhosis of the liver, hepatic carcinoma including adenocarcinoma; acute and chronic pancreatitis; pancreatic carcinoma; portal hypertension; esophageal varicies, etc.

5.3 Methods of Treatment

The present invention provides for a method of treating an ethanol-related disorder comprising administering, to a subject in need of such treatment, an effective amount of a PLD inhibitor.

A subject may be a human or a non-human subject having a PLD enzyme. A subject may be in need of such treatment if the subject has ingested or otherwise had ethanol intake, and/or the subject expects to ingest ethanol and/or the subject regularly ingests ethanol and/or the subject suffers from an ethanol-related disorder and/or the subject is addicted to ethanol (is an alcoholic).

“Treatment” or “treating” refers to an amelioration of the severity and/or duration of an ethanol-related disorder, for example an amelioration of the severity and/or duration of a negative or toxic effect of ethanol.

Accordingly, in particular non-limiting embodiments, the present invention provides for a method of decreasing a negative effect of ethanol on behavior comprising administering, to a subject in need of such treatment, an effective amount of a PLD inhibitor. Such methods may be useful in the management of acute intoxication as well as in rehabilitation from alcohol addiction. “Decreasing a negative effect” means, as that term is used herein decreasing the severity and/or the duration of a negative effect caused by ethanol intake. In a specific, non-limiting embodiment, negative effects of ethanol in a human may be assessed using a sobriety test, for example, the Standardinzed Field Sobriety Test or another test described in one of the following publications which are incorporated herein by reference: Stuster et al., 2006, Hum. Factor 48(3):608-614; Anderson, E. W. and Burns, M. (1997). Standardized Field Sobriety Tests: A Field Study. Proceedings of the 14th International Conference on Alcohol, Drugs and Traffic Safety Volume 2, 635-639; Arend, R., Dioquino, T., Burns, M., Fiorentino, D., Brown, T., Gguyen, S., and Seymour, C. (1999). A Florida Validation Study of the Standardized Field Sobriety Test (SFST) Battery. Department of Transportation, State of Florida; Aschan, G. (1958). Different types of alcohol nystagmus. Acta Otolaryngology, Supplement 140, 69-78; Burns, M., Fiorentino, D., and Stuster, J. (2000). The Observational Threshold Of Horizontal Gaze Nystagmus. In, Proceedings of the International Council on Alcohol, Drugs, and Traffic Safety, Stockholm, Sweden, May; Burns, M. and Anderson, E. W. (1995). A Colorado Validation Study of the Standardized Field Sobriety Test (SFST) Battery. Colorado Department of Transportation; Burns, M. and Moskowitz, H. (1977). Psychophysical Tests for DWI Arrest. U.S. Department of Transportation, National Highway Traffic Safety Administration, DOT-HS-5-01242, Washington, D.C.; Harris, D. H., Dick, R. A., Casey, S. M., and Jarosz, C. J. (1980). The Visual Detection of Driving While Intoxicated. U.S. Department of Transportation, National Highway Traffic Safety Administration, DOT-HS-7-1538, Washington; Harris, D. H. (1980). Visual detection of driving while intoxicated. Human Factors, 22(6), 725-732; Lehti, H. M. J. (1976). The effects of blood alcohol concentration on the onset of gaze nystagmus. Blutalkohol, Vol. 13, 411-414; Moskowitz, H., and Robinson, C. D. (1988). Effects of Low Doses of Alcohol on Driving-Related Skills: A Review of the Evidence. U.S. Department of Transportation, National Highway Traffic Safety Administration, DOT-HS-807-280, Washington, D.C.; Pentilla, A., Tenhu, M., and Kataja, M. (1971). Clinical Examination For Intoxication In Cases of Suspected Drunken Driving. Statistical and Research Bureau of TALJA. Iso Roobertinkatu 20, Helsinki, Finland; Stuster, J. and Burns, M. (1998). Validation of the Standardized Field Sobriety Test Battery at BACs Below 0.10. US Department of Transportation, National Highway Traffic Safety Administration, DOT-HS-808-839, Washington, D.C.; Stuster, J. W. (1997). The Detection of DWI at BACs Below 0.10. U.S. Department of Transportation, National Highway Traffic Safety Administration, DOT-HS-808-654, Washington, D.C.; Stuster, J. W. (1993). The Detection of DWI Motorcyclists. U.S. Department of Transportation, National Highway Traffic Safety Administration, DOT-HS-807-839, Washington, D.C.; Tharp, V., Burns, M., and Moskowitz, H. (1981). Development and Field Test of Psychophysical Tests for DWI Arrest. U.S. Department of Transportation, National Highway Traffic Safety Administration, DOT-HS-805-864, Washington, D.C.; Wilkinson, I. M. S., and/or Kime, R., and Purnell, M. (1974). Alcohol and human eye movement. Brain, 97, 785-792.

In further non-limiting embodiments, the present invention provides for a method of inhibiting a toxic effect of ethanol on a cell or tissue, for example, but not limited to, the brain, liver, and/or pancreas and/or a brain cell, a liver cell, and/or a pancreatic cell, comprising administering, to a cell in need of such treatment, an effective amount of a PLD inhibitor. Such methods may be useful in the treatment of acute toxic effects on these tissues and/or the chronic effects of alcohol abuse. A toxic effect on a cell may be recognized using standard tests, including, but not limited to, viability studies such as trypan blue, MTPT staining, and histological staining (e.g., in the liver, hepatocellular swelling).

PLD inhibitors which may be used in the methods discussed in this section are set forth in section 5.1, above.

A PLD inhibitor may be administered by any suitable route known in the art, including, but not limited to, by oral, subcutaneous, intramuscular, intravenous, intrathecal, inhalation, or rectal administration.

In particular, non-limiting embodiments, the PLD inhibitor is FIPI, administered to achieve a concentration in the cerebrospinal fluid of between about 50 and 2500 nM, or between about 250 and 2000 nM, or between about 250 and 1000 nM. Where the PLD inhibitor is not FIPI, the dose ranges for the (non-FIPI) PLD inhibitor may be determined by multiplying the aforesaid dose ranges for FIPI by the ratio of the IC50 of said PLD inhibitor to the IC50 of FIPI, for example, but not by way of limitation, as measured by an assay that measures PLD activity (see, for example, Scott et al., 2009, Nat Chem Biol. February;5(2):108-17; Monovich et al., 2007, Bioorg. Med. Chem. Lett. 17:2310-2311; Lewis et al., 2009, Bioorg. Med. Chem. Letts. 19:1916-1920; or Lavieri et al., 2009, Bioorg. Med. Chem. Lett. 19:2240-2243).

In particular, non-limiting embodiments, a PLD inhibitor may be administered once or more daily, once or more weekly, or once or more monthly. Periods of treatment may be continuous or discontinuous.

In related, non-limiting embodiments, abstention from alcohol (or inabstinence) of a subject may be evaluated by detecting and/or measuring PEtOH in a sample from the subject, where the presence of PEtOH in the sample indicates that the subject has been exposed to (e.g., ingested) alcohol. The sample may be a blood sample or a tissue sample (e.g. a liver sample) or a urine sample. In non-limiting embodiments, PEtOH may be detected and/or measured by LC-MS.

### 6. EXAMPLE 1

Despite the burden of alcoholism for our society, available treatments are limited and many patients relapse, showing that new therapeutics are in need. Therefore, understanding the molecular and cellular mechanisms underlying the behavioral effects of ethanol is critical. Phospholipase D (PLD) has been hypothesized to modulate the effects of ethanol. PLD normally produces phosphatidic acid (PA) by hydrolyzing phosphatidylcholine (PC), but in the presence of ethanol generates phosphatidylethanol (PEtOH), a highly stable lipid that rapidly accumulates in the brain, where it may alter the physico-chemical and signaling properties of neuronal membranes. Despite the fact that PLD enzymes are primary targets of ethanol, no experimental evidence supporting a role for this pathway in mediating the effects of this drug has been reported. To test this hypothesis, we have developed a genetic model lacking one of the two main PLD isoforms, PLD2. Using a lipidomics approach relying on liquid chromatography-mass spectrometry, we found that the brains from ethanol-injected mice lacking PLD2 (PLD2KO mice) exhibit half the amount of PEtOH one hour post-injection, while no other lipids, such as PA, PC and diacyglycerol were altered. Blood ethanol levels were comparable for both genotypes. The ataxic effects of ethanol were examined in wild type and Pld2 KO mice using the accelerated rotarod. Trained Pld2 KO mice showed a similar ability to stay on the spinning row relative to wild type mice in the absence of ethanol treatment. Moreover, both genotypes were equally affected by the impairing effect of ethanol shortly after the injections (3 min post injection). In contrast, the recovery of locomotor ability following ethanol intoxication was significantly more pronounced in Pld2 KO mice relative to controls. Specifically, Pld2 KO mice were able to remain for approximately 35% more time on the accelerating rotarod than wild type mice mice one hour post-injection (p<0.05). Our study provides the first genetic evidence for arole of the PLD pathway, and potentially PEtOH, in the molecular and behavioral responses to ethanol.

### 7. EXAMPLE 2

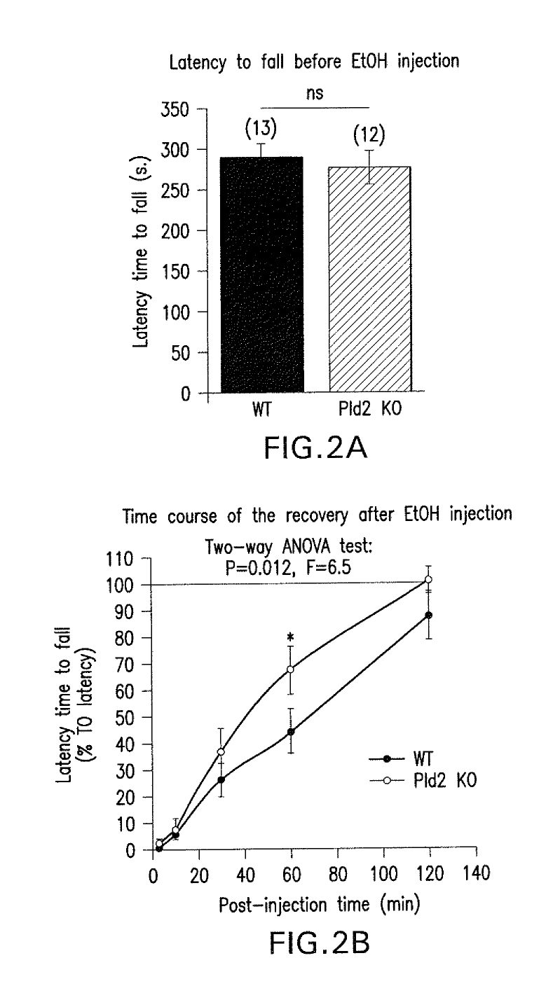

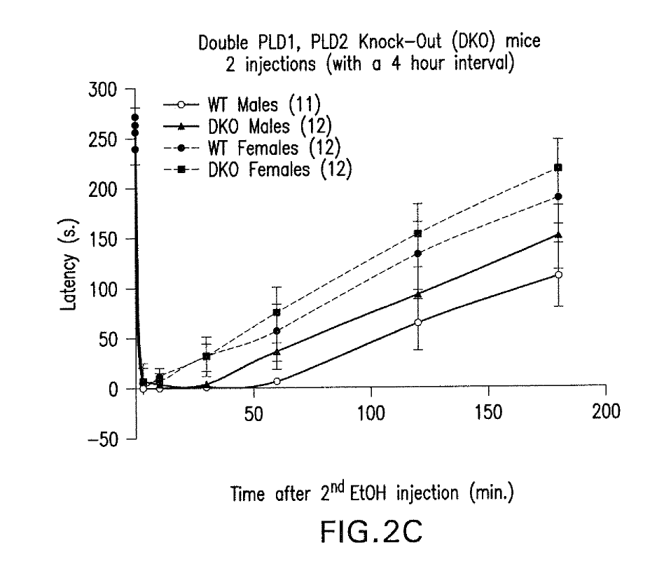

PLD2KO mice were generated as described in Example 3, below. To test the effect of genetic PLD2 ablation on motor performance under the influence of ethanol, the rotarod test was used and applied to wild type and PLD2KO mice. In the test, mice were placed on the rotarod cylinder, the speed of which was gradually accelerated from 3 to 35 revolutions per minute over a 6 minute period. Latency to fall off the cylinder was recorded. On day 1, ethanol naive mice (WT, n=13; PLD2KO, n=12) were trained to remain on the accelerating rotarod (4 trials of 6 minutes). On day 2, the fall latency was measured for the wild type and PLD2KO trained mice (1 trial of 6 minutes). As shown in FIG. 2A, no difference was found between WT and PLD2KO mice. Mice were then injected with ethanol (3 g/kg i.p.), and tested for recovery of balance on the accelerating rotarod at 3, 30, 60 and 120 minutes after injection.1. As shown in FIG. 2B, PLD2KO mice recovered faster from ethanol injections in the rotarod test.

FIG. 2C presents the rotarod test results for mice that are double knockouts for PLD1 and PLD2. The post-injection recovery is faster in mice lacking both PLD1 and PLD2 relative to controls. The latency to fall from the accelerating rotarod was measured at 3, 10, 30 and 60 min after 2 intraperitoneal ethanol injection separated by approximately 4 hours in the following genotypes: WT males (n=11) and females (n=12), Pld1−//Pld2−/− males (n=12) and females (n=12).

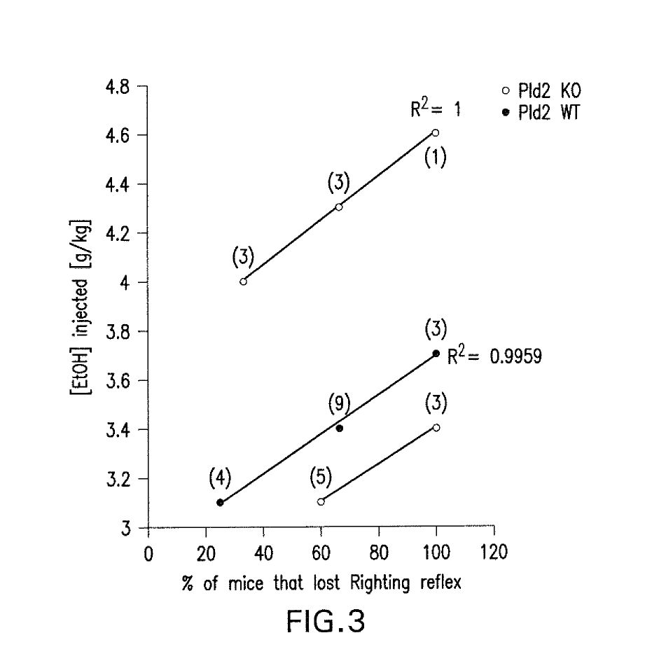

Similarly, in a different test of motor ability which measures the righting reflex, acute intoxication of mice with various concentrations of ethanol (via i.p.) was found to lead to a loss of righting reflex in a smaller fraction of PLD2 knockout mice relative to controls (FIG. 3). Higher doses of ethanol were required in the Pld2 knockout mice to achieve the same level of impairment as was observed in the control mice.

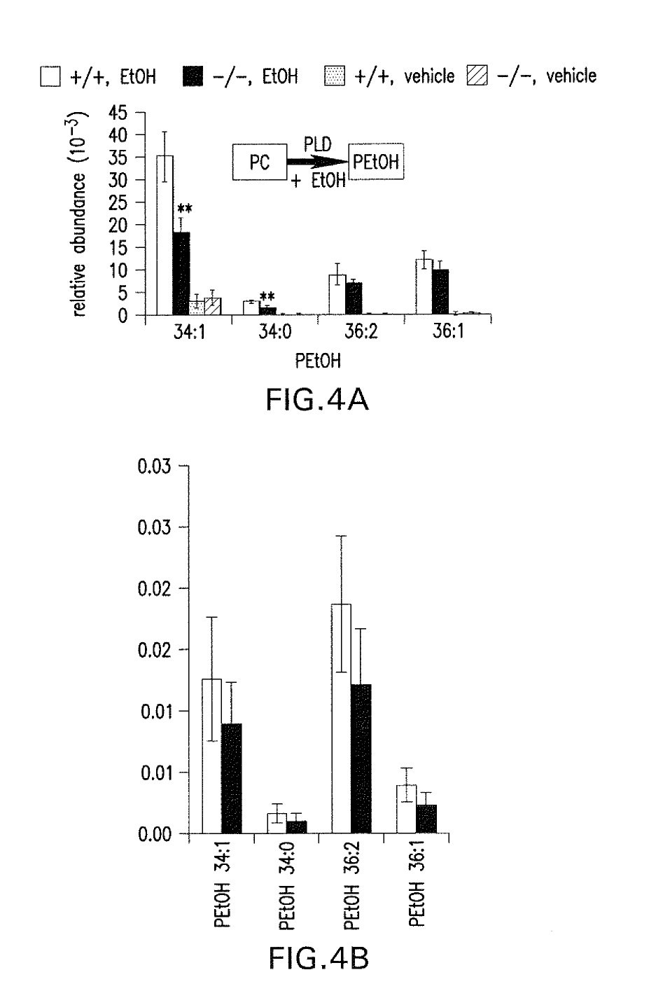

To provide an anatomical/pharmacologic correlation with the above observations on motor ability, the level of phosphatidylethanol was measured in the brain and liver of PLD2KO mice relative to controls based on mass spectrometry experiments. As shown in FIG. 4A-B) PLD2KO mice exhibited less PLD activity in the brain (FIG. 4A) and liver (FIG. 4B) relative to wild-type mice.

### 8. EXAMPLE 3

### 8.1 Materials and Methods

Generation of PLD2KO Mice.

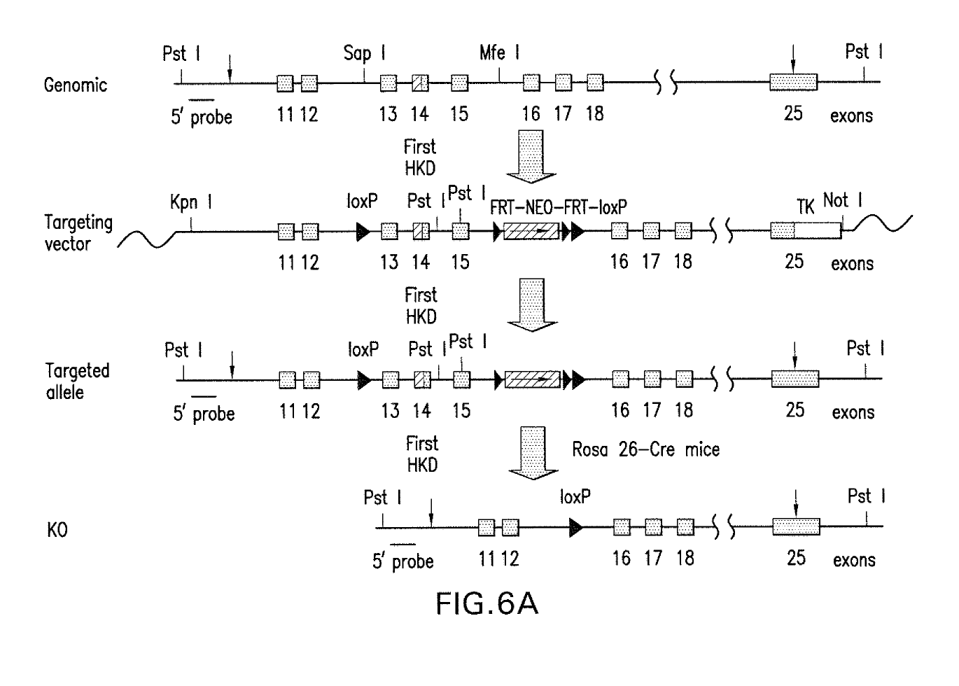

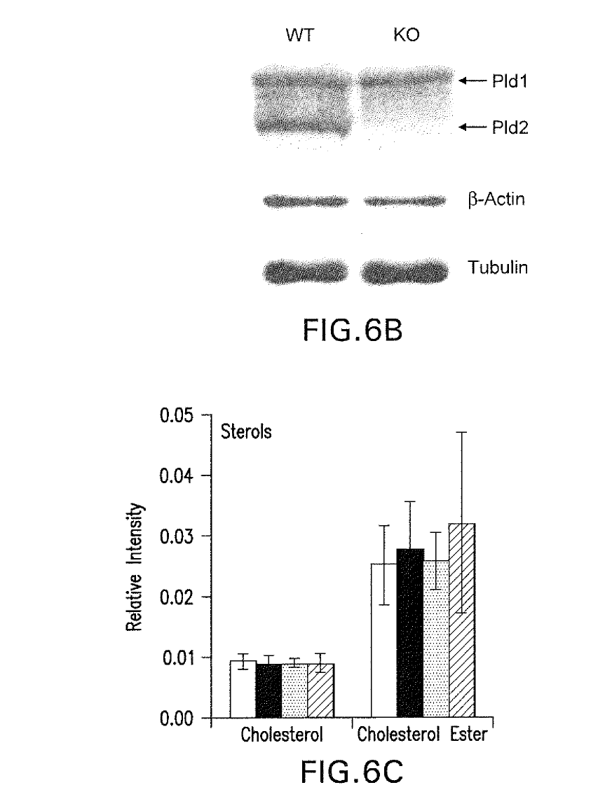

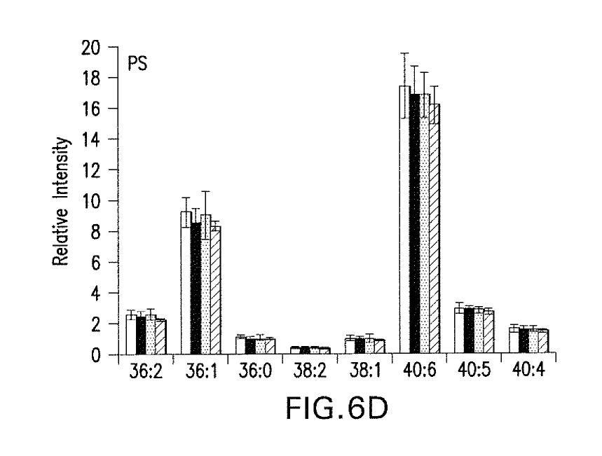

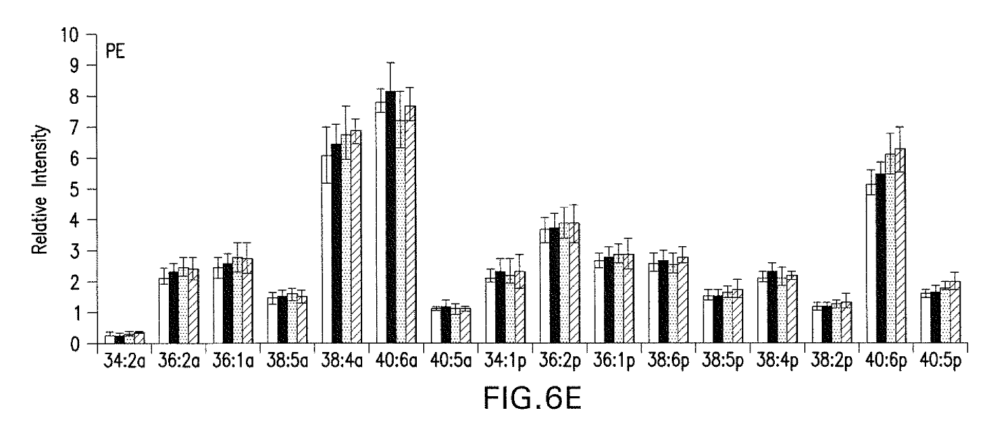

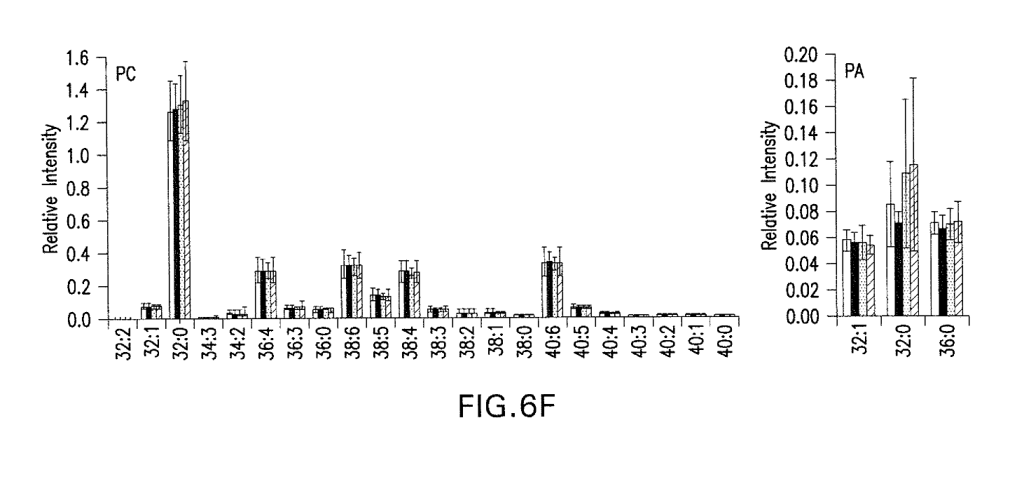

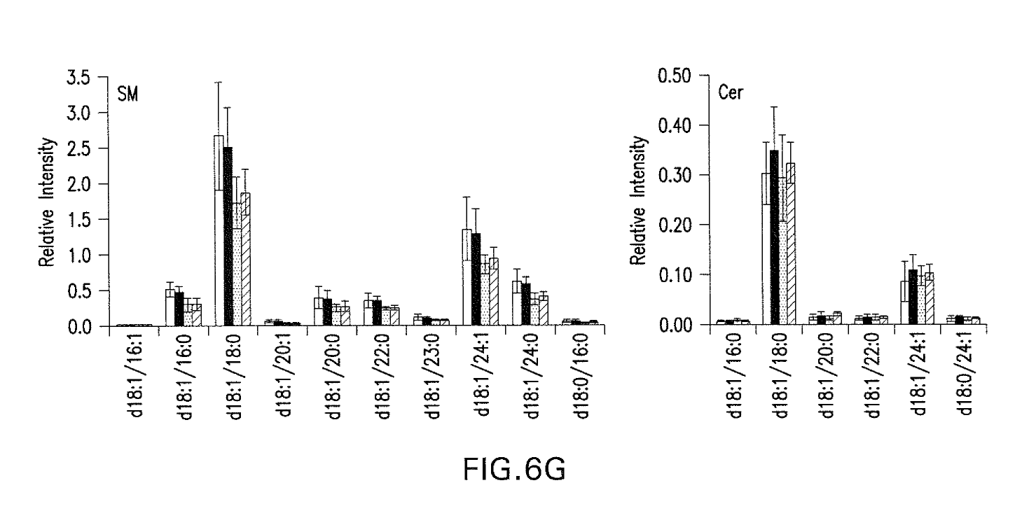

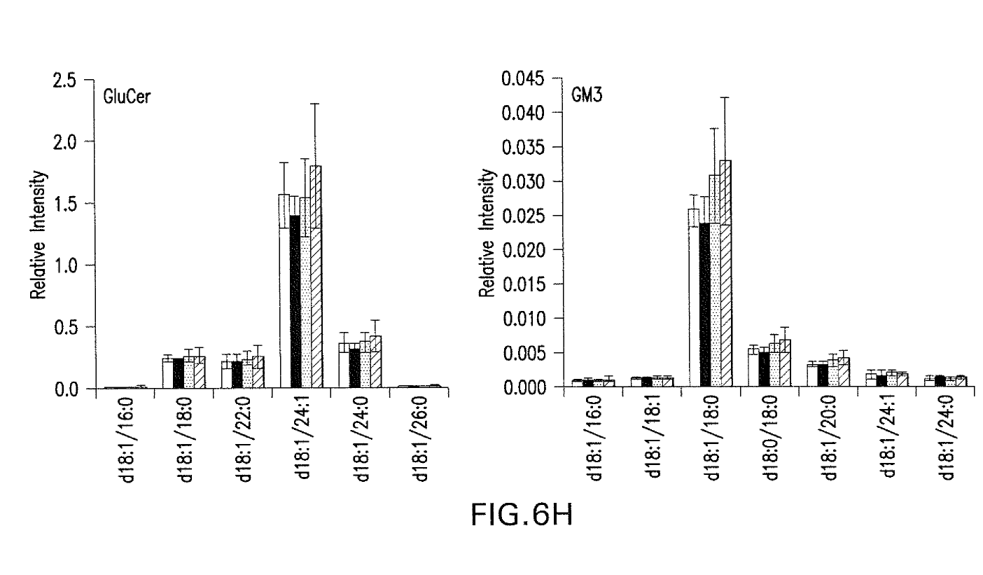

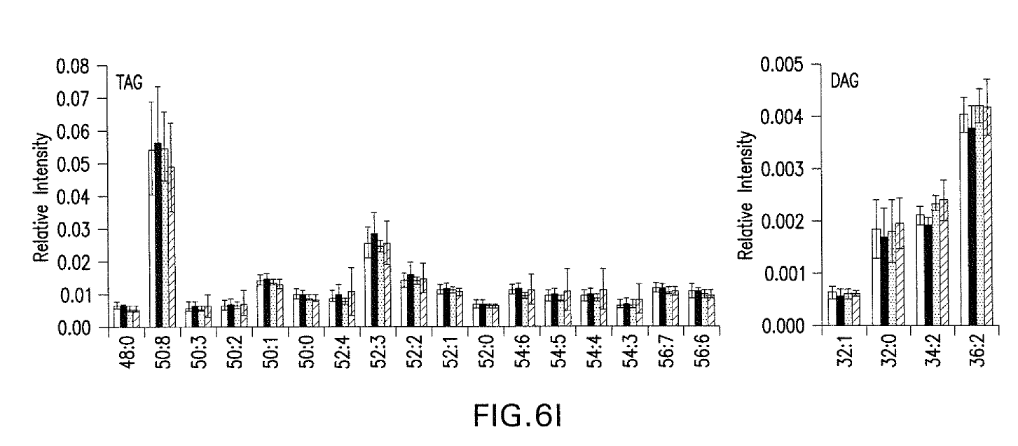

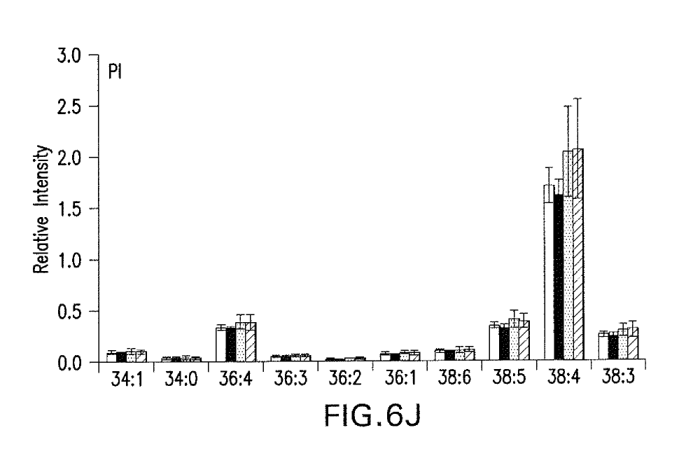

As shown in FIG. 6A, an FRT-NEO-FRT-loxP cassette was inserted downstream of exon 15 from the Pld2 gene at the SspI site and the second loxP sequence was subcloned upstream of exon 13 at the MfeI site. Exon 14 contains the sequence encoding the first “HKD” motif of PLD2, which is essential for the catalytic activity of PLD2. Pld2Flox Neo/+ mice were bred with a “deleter” strain of mice expressing Cre recombinase (Rosa26) to eliminate exons 13-15 and produce Pld2+/− mice. Pld2+/− mice were then intercrossed to create Pld2+/+ and Pld2−/− mice. The genetic background of the animals is mixed (C57B16, 129SVJ). Homologous recombination was verified by Southern blot and PCR analysis. Western blot analysis was performed using standard ECL procedures, using a pan-PLD antibody.

Lipid Analysis.

Mice brain lipid extracts were spiked with appropriate internal standards and analyzed by LC-MS operated in multiple reactions monitoring mode (4), Polar glycerophospholipids (including PEtOH) and sphingolipids and non-polar neutral lipids were separated using normal phase (5) and reverse phase (4) HPLC respectively. PEtOH levels were referenced to synthetic PEtOH 32:0 which was added in excess of endogenous levels of this species. PEtOH measurements were made by following the parent. 181 m/z product ion transition produced by a constant collision energy at −45 eV. The remaining lipid classes were measured using previously reported MRM transition pairs and instrument settings (4).

Behavioral Study.

The rotarod (Basile automated rotarod, accelerating from 3 to 35 revolutions per min over a period of 300 s.) consisted of a plastic roller (3 cm in diameter) with small grooves running along its turning axis. The latency to fall off the rotarod was recorded. On day 1, ethanol-naïve mice were trained to remain on the rotarod by giving them 4 trials of 6 min each. The next day, one single trial was performed before injecting the animals with ethanol (3 g/kg i.p.), mice were tested for recovery of balance at 3 and 60 min after injection. Animals staying for 360 s were removed from the rotarod and recorded as 300 s. WT and Pld2−/− mice were 2-6 month old (WT: 9 females, 7 males; Pld2−/−: 8 females, 7 males).

Alcohol Measurements.

Blood was obtained following decapitation and mixed with 3.4% perchloric acid. The mixture was immediately vortexed and centrifuged. Aliquots of the supernatant (50 μl) were incubated with 3 ml of assay buffer [semicarbazide HCl buffer (67 mM); sodium pyrophosphate (67 mM) and glycine (20 mM), pH 8.5] containing NAD (1 mg) and yeast alcohol dehydrogenase (170 units) for 1 h at room temperature. The absorption coefficient was measured at 340 nm. The ethanol standards (0.1-3 mg) were processed similarly along with the samples.

Statistical Analysis.

Statistical analysis was performed using Student's t test unless otherwise indicated.

### 8.2 Results and Discussion

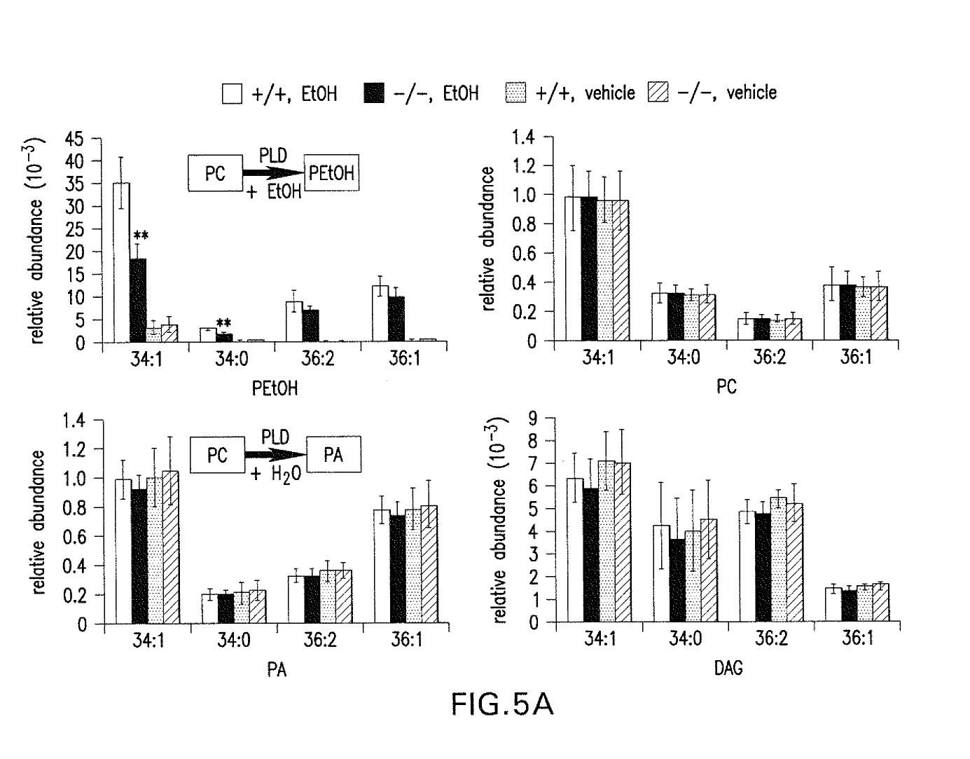

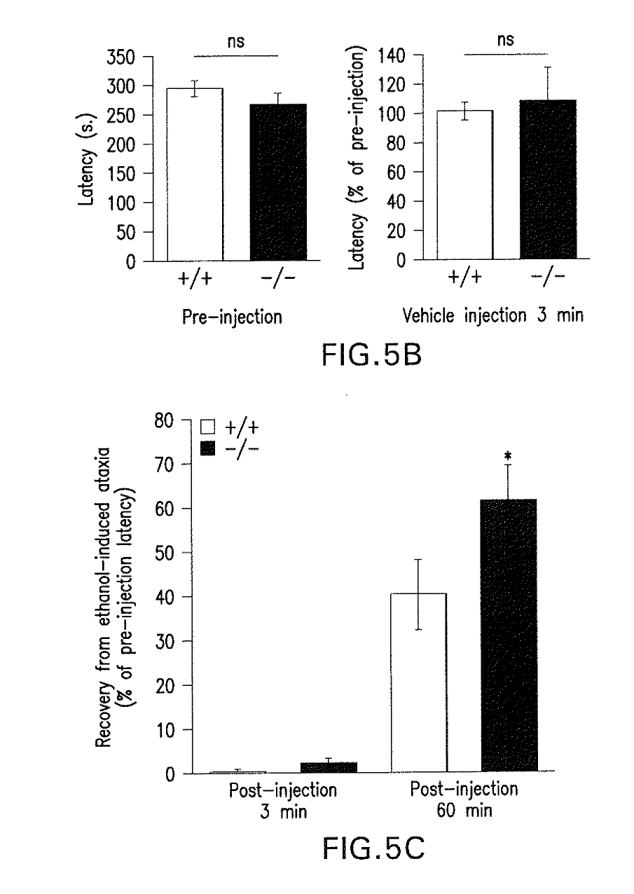

A genetic model was developed lacking one of the two main PLD isoforms, PLD2, which is expressed in all tissues, including the brain, and concentrated at the plasma membrane and endosomal compartment of various cell types (FIG. 6A)(3). Western blot analysis using specific antibodies showed that the PLD2 immunoreactivity is absent in adult whole brain tissue derived from Pld2−/− (PLD2KO)mice (FIG. 6B), which do not exhibit any phenotypes at the organismal level and, importantly, no alteration of body weight (Pld2+/+: 29±1 g; Pld2−/−: 31±1 g; p=0.3, age range: 2-6 months). The effect of ethanol on brain lipids was investigated in Pld2+/+ and Pld2−/− mice, which were subjected to intraperitoneal (i.p.) injections of either ethanol (3 g/kg) or vehicle and sacrificed 60 min after these treatments. Total brain lipids were subsequently analyzed via liquid chromatography-mass spectrometry (LC-MS) (4, 5). Injection of ethanol, but not vehicle, led to the production of PEtOH in brain tissue from both genotypes (FIG. 5A). However, the PLD activity was significantly decreased in the brain of Pld2−/− (PLD2KO) mice as evidenced by the ˜50% reduction in the levels of the most abundant PEtOH species (34:1) (FIG. 5A, p<0.01). Comparable results or trends were found for 34:0 PEtOH (P<0.01) and 36:2/36:1 PEtOH species, respectively. There was no effect of genotype or ethanol treatment on the levels of PLD's main physiological substrate (PC), enzymatic reaction product (PA) and potential metabolite thereof (diacylglycerol, DAG) (FIG. 5A). Strikingly, the lipid profiles of the two genotypes were comparable in the presence or absence of alcohol treatment, as tested by comprehensive lipidomic analysis covering most membrane lipid classes, i.e. glycerophospholipids (PC, PS, PI, PE), sphingolipids (SM, Cer, GluCer, and GM3) and cholesterol, as well as major neutral lipids such as TAG, DAG, and cholesterol esters, all including predominant fatty acyl combinations (FIGS. 5A and 6J). Thus, the only detectable lipid change in the brain of Pld2−/− (PLD2KO) mice is a deficiency of PEtOH following ethanol intoxication.

We next explored whether the loss of Pld2 altered the behavioral effects of ethanol. We tested the ability of wild type and Pld2−/− (PLD2KO) mice to remain on an accelerating rotarod (6), a common paradigm to test the effect of ethanol intoxication. Trained Pld2−/− mice showed a similar ability to stay on the spinning row relative to wild type mice in the absence of ethanol treatment, demonstrating that the motor skills and motor learning is not impaired in the mutant mice (FIG. 5B). Next, the recovery of locomotor ability following ethanol intoxication (3 g/kg via i.p.) was monitored in wild type and Pld2−/− mice. While both genotypes were equally affected by the impairing effect of ethanol shortly after the injections (3 min), the recovery of Pld2−/− mice was significantly more pronounced than that of wild type mice at 60 min post-injection, where Pld2−/− mice were able to remain for approximately 35% more time on the accelerating rotarod than controls (p<0.05) (FIG. 5C). Blood ethanol levels were comparable for both genotypes one hour post-injection (1.60±0.24 and 1.79±0.31 mg/ml for Pld2+/+ and Pld2−/− mice, resp.; n=5; p>0.05) indicating that changes in the systemic metabolism of ethanol do not underlie the differential effects of ethanol.

This study provides the first genetic evidence for a role of the PLD pathway in the molecular and behavioral responses to ethanol. Mice lacking PLD2 recover more efficiently from ethanol-induced ataxia in acute intoxication experiments, and this behavioral change correlates with a significant decrease in brain-associated PLD activity and specifically, reduced brain levels of PEtOH in these animals. The finding that no other lipid changes are observed in these mice upon alcohol treatment (including no detectable changes in the physiological enzymatic products of PLD, PA) is consistent with the notion that brain PEtOH levels may modulate the behavioral effects of ethanol. In agreement with this idea, PEtOH has been shown to affect the physicochemical properties of cellular membranes (e.g. fluidizing effect) with potentially major implications on their permeability as well as to interfere with intracellular signaling cascades (e.g. PKC pathway) (1). It is also possible that a subthreshold decrease in PA synthesis resulting from ethanol treatment may also contribute to the increased resistance of Pld2−/− mice to this drug. Pld2−/− mice thus afford the opportunity to study the role of the PLD pathway and PEtOH in the pleiotropic effects of alcohol, including the addictive effects of this drug, since PEtOH persists in the brain long after exposures to ethanol. Finally, based on its ability to modulate responses to ethanol, the PLD pathway is a target for novel therapeutics in the treatment of alcoholism.

### 9. REFERENCES

1. P. M. Newton, R. O. Messing, Pharmacol Ther 109, 227 (January, 2006).
2. L. Gustaysson, Alcohol Alcohol 30, 391 (July, 1995).
3. G. M. Jenkins, M. A. Frohman, Cell Mol Life Sci 62, 2305 (October, 2005).
4. R. Chan et al., J Viral 82, 11228 (November, 2008).
5. T. R. Pettitt, M. McDermott, K. M. Sagib, N. Shimwell, M. J. Wakelam, Biochem J 360, 707 (Dec. 15, 2001).
6. N. R. Rustay, D. Wahlsten, J. C. Crabbe, Proc Natl Acad Sci USA 100, 2917 (Mar. 4, 2003).

Various publications are cited herein, the contents of which are hereby incorporated by reference in their entireties.

We claim:

1. A method of treating an ethanol-related disorder comprising administering, to a subject in need of such treatment, an effective amount of a phospholipase D inhibitor, wherein the ethanol-related disorder is selected from the group consisting of impaired motor ability, impaired coordination, ataxia, impaired cognition, vertigo, nausea, anterograde amnesia; substance abuse, addiction, symptoms and signs of detoxification, neurodegeneration, cerebellar degeneration, seizure, hypertension, cardiovascular disease, cardiomyopathy, cardiac angina, myocardial infarction, vascular disease, cerebral ischemia, cerebral infarction, acute hepatitis, chronic hepatitis, fatty liver, hepatic fibrosis, cirrhosis of the liver, hepatic carcinoma, acute pancreatitis, chronic pancreatitis, pancreatic carcinoma, portal hypertension, and esophageal varicies.

2. A method of decreasing a negative effect of ethanol on behavior comprising administering, to a subject in need of such treatment, an effective amount of a phospholipase D inhibitor.

3. A method of inhibiting a toxic effects of alcohol on a cell or tissue comprising administering, to a cell in need of such treatment, an effective amount of a phospholipase D inhibitor.

4. The method of claim 2, where the negative effect of ethanol on behavior is selected from the group consisting of impaired motor ability, impaired coordination, ataxia, impaired cognition, vertigo, nausea, anterograde amnesia; substance abuse, addiction, and symptoms and signs of detoxification.

5. The method of claim 3, wherein the cell or tissue is selected from the group consisting of brain, liver, pancreas, a brain cell, a liver cell, and a pancreatic cell

6. The method of claim 1 where the phospholipase D inhibitor is a phospholipase D2 inhibitor.

7. The method of claim 1 where the phospholipase D inhibitor is a halopemide derivative.

8. The method of claim 1 where the phospholipase D inhibitor is selected from the group consisting of phospholipase D inhibitors depicted in FIG. 7A-M.

9. The method of claim 1 where the phospholipase D inhibitor is 5-Fluoro-2-indolyl des-chlorohalopemide (“FIPI”).

10. The method of claim 2 where the phospholipase D inhibitor is a phospholipase D2 inhibitor.

11. The method of claim 2 where the phospholipase D inhibitor is a halopemide derivative.

12. The method of claim 2 where the phospholipase D inhibitor is selected from the group consisting of phospholipase D inhibitors depicted in FIG. 7A-M.

13. The method of claim 2 where the phospholipase D inhibitor is 5-Fluoro-2-indolyl des-chlorohalopemide (“FIPI”).

14. The method of claim 3 where the phospholipase D inhibitor is a phospholipase D2 inhibitor.

15. The method of claim 3 where the phospholipase D inhibitor is a halopemide derivative.

16. The method of claim 3 where the phospholipase D inhibitor is selected from the group consisting of phospholipase D inhibitors depicted in FIG. 7A-M.

17. The method of claim 3 where the phospholipase D inhibitor is 5-Fluoro-2-indolyl des-chlorohalopemide (“FIPI”).

18. A method of monitoring phospholipase D activity comprising (i) administering ethanol to a cell, tissue or subject and (ii) measuring phosphatidylethanol in a sample from the cell, tissue or subject by subjecting the sample to mass spectrometry or liquid chromatography-mass spectrometry, wherein the quantity of phosphatidylethanol reflects the phospholipase D activity present in the cell, tissue, or subject.
